(19) United States
(12) Patent Application Publication
(10) Pub. No.: US 2009/0163757 A1
(43) Pub. Date: Jun. 25, 2009

(54) Linear olefin isomer isomerization using molecular sieve catalysts

(76) Inventor: Jeffery C. Gee, Kingwood, TX (US)

Correspondence Address: CHEVRON PHILLIPS CHEMICAL COMPANY LP, LAW DEPARTMENT - IP, P.O BOX 4910, THE WOODLANDS, TX, 77387-4910, US

(21) Appl. No.: 12/004,216

(22) Filed: Dec. 20, 2007

(51) Int. Cl. C07C 5/22

(52) U.S. Cl. 585/671

(57) ABSTRACT

The present disclosure describes methods for isomerizing olefins which produce isomerized products having low levels of skeletal isomerization. The methods use a combination of molecular sieve catalyst and isomerization reaction temperature, and weight hourly space velocity to achieve the low levels of skeletal isomerization.

### BACKGROUND OF THE INVENTION

Alpha olefins are common articles of commerce and precursors to other articles of commerce such as polymers, detergents, and synthetic fluids, among others uses. Internal olefins are also utilized as precursors to articles of commerce as detergents and additives for producing paper, among other uses. However, while alpha olefin are readily available in carbon numbers from 3 to greater than 30, internal olefins are commercially available in large quantities from refinery streams for carbon numbers lower than 10. Even when internal olefins are commercially available, they may contain significant amounts of branched olefins and/or paraffins that can be difficult to remove when linear internal olefins are desired. In these instances, linear alpha olefins are isomerized to internal olefins.

A common method of isomerizing linear alpha olefins utilizes inorganic materials such as iron pentacarbonyl or rhodium trichloride. While these processes efficiently produce linear internal olefins with low levels of branched olefins, the removal of the inorganic agents can be difficult, complicate the process of producing the linear internal olefins, and increase the cost of operating processes to isomerize the linear alpha olefins.

Alternate processes for isomerizing linear alpha olefin that use solid catalysts such as molecular sieves, zeolites, or aluminas have been reported. The solid catalyst processes have an advantage that the isomerized olefin is easy to separate from the isomerization catalyst. However, these processes are prone to skeletally isomerizing the olefins to produce significant quantities of branched olefins. Processes for isomerizing olefins using solid catalysts (e.g. molecular sieves) which produce low levels of skeletally isomerized olefins are needed.

### SUMMARY OF THE INVENTION

Disclosed herein are methods for isomerizing olefins to produce an isomerized product having a limited quantity of skeletally isomerized olefins. In an aspect, the method for producing the isomerized olefin comprises contacting an olefin feedstock with a molecular sieve catalyst and isomerizing the olefin in a reactor at reaction conditions capable of producing an olefin reactor effluent comprising an isomerized product having a limited quantity of skeletally isomerized olefin.

In an embodiment, the isomerization methods disclosed herein isomerize a linear alpha olefin to an olefin reactor effluent comprising less than 10 weight percent linear alpha olefin and an isomerized product having a limited quantity of skeletally isomerized olefin. In an embodiment, the reactor effluent contains less than 10 weight percent skeletally isomerized olefin; or alternatively less than 5 weight percent skeletally isomerized olefin.

In an embodiment, the reaction conditions capable of isomerizing the olefins comprise a weight hourly space velocity ranging from 0.01 to 1.0; or alternatively, a temperature ranging form 80 to 220° C. and a weight hourly space velocity ranging from 0.02 to 0.7. In another embodiment, the reaction conditions capable of isomerizing the olefin to an isomerized product having a limited quantity of skeletally isomerized olefins comprise a weight hourly space velocity ranging from 0.04 to 0.18; or alternatively, a temperature ranging form 80 to 220° C. and a weight hourly space velocity ranging from 0.04 to 0.18.

In an embodiment, the molecular sieve catalysts which isomerize the olefin to an isomerized product having a limited quantity of skeletally isomerized olefins comprise pores having a pore size ranging form 4 to 8 angstroms. In another embodiment, the molecular sieve catalysts which isomerize the olefin to an isomerized product having a limited quantity of skeletally isomerized olefins comprise oval one-dimensional pores having a minor axis ranging from 2 to 6 angstroms and a major axis ranging group 3 to 9 angstroms. In some embodiments, the molecular sieve catalysts which isomerize the olefin to an isomerized product having a limited quantity of skeletally isomerized olefin, may be a SAPO, SSZ, or ZSM molecular sieve.

### DEFINITIONS

The term “hydrocarbon(s)” and its derivatives (e.g. “hydrocarbyl”) whenever used in this specification and claims refer to compounds or groups comprising only hydrogen and carbon. The term “hydrocarbon” may also be prefaced with other descriptors that further limit the scope of the term. For example “olefinic hydrocarbons” refer to compounds or groups containing only hydrogen and carbon and have at least one olefinic double bond, “aromatic hydrocarbon(s)” refer to compound or groups containing only hydrogen and carbon and having an aromatic ring or ring system (i.e. a benzene ring or naphthalene ring system, among others), and “saturated hydrocarbon(s)” refers to compounds or groups containing only hydrogen and carbon and having no olefinic or aromatic double bonds. The term “hydrocarbon(s)” when prefaced with an atom or functional group descriptor indicates that the compound(s) or group(s) contains only hydrogen, carbon, and the indicated atom or functional group. For example, a “halogenated hydrocarbon(s)” refers to a compound(s) containing hydrogen, carbon, and at least one halogen atom but no other type of heteroatom.

The term “isomerization” whenever used in this specification and claims refers to processes wherein the olefin double bond of an olefin changes position along the backbone of the olefin, and/or a rearrangement of carbon atoms has occurred. The term “isomerized product” refers to a product wherein the olefin double bond has changed its position and/or a rearrangement of carbon atoms has occurred.

The term “olefin isomerization” and its derivatives whenever used in this specification and claims refers to processes wherein the olefin double bond of an olefin changes position along a carbon backbone of an olefin. The term “isomerized olefin” and its derivatives refers to a product wherein the olefin double bond has changed its position. The term “isomerized olefin” excludes material in which the olefin double bond has not changed its position. The terms “olefin isomerization” and “isomerized olefin” and their derivatives may be utilized with other terms to further describe the particular “olefin isomerization” and “isomerized olefin.” For example “alpha olefin isomerization” and “hydrocarbon olefin isomerization” refer to a process(es) for isomerizing an alpha olefin and hydrocarbon olefin, respectively, while “isomerized alpha olefin” and “isomerized hydrocarbon” refer to a product wherein the double bond of an alpha olefin or hydrocarbon olefin, respectively, has changed position.

The term “skeletal isomerization” and its derivatives whenever used in this specification and claims refer to processes wherein a rearrangement of carbon atoms has occurred. The rearrangement may create new branches on the backbone of the olefin and/or be the result of a movement of a branch along a carbon backbone. The term “skeletal isomerized product” and its derivatives refers to a product wherein a rearrangement of carbon atoms has occurred. The terms “skeletal isomerization” and “skeletal isomerized product” and their derivatives may be utilized with other terms to further describe the particular “skeletal isomerization” and “skeletal isomerized product.” For example, “skeletal olefin isomerization” and “skeletal hydrocarbon isomerization” refer to processes for skeletally isomerizing an olefin and hydrocarbon, respectively, while “skeletally isomerized olefin” and “skeletally isomerized hydrocarbon” and their derivatives refer to products wherein the carbon atoms of an olefin and hydrocarbon, respectively, have been rearranged. It should be noted that the terms “skeletal olefin isomerization” and “skeletal isomerized olefin” do not indicate whether or not an olefin isomerization process has occurred (i.e. whether the olefin double bond has changed position). It should also be noted that a branched olefin which has undergone a shift in the position of the double bond is not a skeletally isomerized olefin unless there has also been a rearrangement of carbon atoms.

It should be noted that a particular olefin molecule may undergo both olefin and skeletal isomerization. Consequently, a particular olefin molecule which has been isomerized may contribute to the isomerized olefin content and the skeletally isomerized olefin content of an isomerized product.

The terms “feedstock olefin(s)” or “olefin feedstock” whenever used in this specification and claims refer to the olefinic compounds which are originally present in the feedstock composition. The terms “feedstock olefin(s)” or “olefin feedstock” do not include any new olefinic compounds produced by olefin or skeletal isomerization.

The term “alpha olefin” whenever used in this specification and claims refers to an olefin that has a double bond between the first and second carbon atom. The term “alpha olefin” includes linear and branched alpha olefins unless expressly stated otherwise. In the case of branched alpha olefins, a branch may be at the 2-position (a vinylidene) and/or the 3-position or higher with respect to the olefin double bond. The term “vinylidene” whenever used in this specification and claims refers to an alpha olefin having a branch at the 2-position with respect to the olefin double bond. The term “alpha olefin,” by itself, does not indicate the presence or absence of heteroatoms and/or the presence or absence of other carbon-carbon double bonds unless explicitly indicated. The term “hydrocarbon alpha olefin” or “alpha olefin hydrocarbon” refers to alpha olefin compounds containing only hydrogen and carbon.

The term “normal alpha olefin” whenever used in this specification and claims refers to a linear hydrocarbon mono-olefin having a double bond between the first and second carbon atom. It should be noted that “normal alpha olefin” is not synonymous with “linear alpha olefin” as the term “linear alpha olefin” can include linear olefinic compounds having a double bond between the first and second carbon atoms and having heteroatoms and/or additional double bonds.

The term “consists essentially of normal alpha olefin(s),” or variations thereof, whenever used in this specification and claims refers to commercially available normal alpha olefin product(s). The commercially available normal alpha olefin product can contain non-normal alpha olefin impurities such as vinylidenes, internal olefins, branched alpha olefins, paraffins, and diolefins, among other impurities, which are not removed during the normal alpha olefin production process. One of ordinary skill in the art will recognize that the identity and quantity of the specific impurities present in the commercial normal alpha olefin product will depend upon the source of commercial normal alpha olefin product. Consequently, the term “consists essentially of normal alpha olefins” and its variants is not intended to limit the amount/quantity of the non-linear alpha olefin components any more stringently than the amounts/quantities present in a particular commercial normal alpha olefin product unless explicitly indicated.

One source of commercially available alpha olefins products is the oligomerization of ethylene. A second source of commercially available alpha olefin products is Fischer-Tropsch synthesis streams. One source of commercially available normal alpha olefin products produced by ethylene oligomerization which may be utilized as an olefin feedstock is Chevron Phillips Chemical Company LP, The Woodlands, Tex. Other sources of commercially available normal alpha olefin products produced by ethylene oligomerization which may be utilized as an olefin feedstock include Ineos Oligomers (Feluy, Belgium), Shell Chemicals Corporation (Houston, Tex. or London, United Kingdom), Idemitsu Kosan (Tokyo, Japan), and Mitsubishi Chemical Corporation (Tokyo, Japan), among others. One source of commercially available normal alpha olefin products produced, and optionally isolated from Fisher-Tropsch synthesis streams, includes Sasol (Johannesburg, South Africa), among others.

The term “internal olefin(s)” whenever used in this specification and claims refers to an olefin which has a double bond at any position other than between the first and second carbon atoms. An “internal olefin(s)” can be linear or branched. A “branched internal olefin” may have a branch attached to one of the carbon atoms of the internal double bond and/or may have a branch at any carbon atom other than those participating in the internal olefin double bond. The term “internal olefin(s)” does not indicate the presence or absence of other groups, branches, heteroatoms, or double bonds within the “internal olefin(s)” unless explicitly indicated.

The term “reactor effluent” generally refers to all the material which exits the reactor. The term “reactor effluent” may also be prefaced with other descriptors that limit the portion of the reactor effluent being referenced. For example, the term “olefin reactor effluent” refers to the effluent of the reactor which contains an olefin (i.e. carbon-carbon) double bond.

### DETAILED DESCRIPTION

The present disclosure relates to methods for producing isomerized olefins. Generally, the disclosure relates to a method for producing an isomerized olefin product having particular features. Minimally, the method for producing isomerized olefins comprises: a) contacting an olefin feedstock and a molecular sieve catalyst; and b) isomerizing the olefins in a reactor at reaction conditions effective for isomerizing the olefin feedstock to form a olefin reactor effluent. In an embodiment, the olefin reactor effluent may comprise non-isomerized olefin and isomerized product. The isomerized product may comprise, or consist essentially of, isomerized olefin and/or skeletally isomerized olefins. In another embodiment, the olefin reactor effluent may comprise, or consist essentially of, non-isomerized olefins, isomerized olefins, and/or skeletally isomerized olefins.

Features of the method(s) such as the olefin feedstock, features of the olefins of the olefin feedstock (if any), molecular sieve catalyst, features of the molecular sieve catalyst (if any), the olefin reactor effluent, features of the olefin reactor effluent (if any), the isomerized olefin, features of the isomerized olefin (if any), the skeletally isomerized olefins, features of the skeletally isomerized olefin (if any), isomerization reaction conditions, constraints on the isomerization reaction conditions, and other process/method features and/or steps are independently described herein. These features can be utilized in any combination necessary to describe the method(s) for producing isomerized olefins.

Generally, the olefin feedstock can comprise, or consist essentially of, any olefinic compound. Further features that can be utilized to describe the olefins of the olefin feedstock may include the type of olefins present, the carbon number of the olefins present, and/or the content of a type(s) of olefins present (i.e. weight percent or mole percent), among other olefin feedstock features described herein. These features of the olefin feedstock are independently described herein and may be utilized in any combination to describe the olefins of the olefin feedstock.

In an embodiment, the olefins of the olefin feedstock can comprise, or alternatively consist of aliphatic olefins. In some embodiments, the olefins of the olefin feedstock can comprise, or consist essentially of, linear olefins, branched olefins, or combinations thereof, alternatively, linear olefins; or alternatively, branched olefins. In other embodiments, the olefins of the olefin feedstock (whether aliphatic, linear or branched, or combinations thereof) can comprise, or consist essentially of acyclic olefins. In some embodiments, the olefins of the olefin feedstock (whether aliphatic, linear or branched, acyclic, or combinations thereof) may comprise, or consist essentially of, hydrocarbon olefins. In an embodiment, the olefins of the olefin feedstock (whether aliphatic, linear or branched, acyclic, hydrocarbon, or combinations thereof) may comprise, or consist essentially of, alpha olefins. In some embodiments, the olefins of the olefin feedstock (whether aliphatic, hydrocarbon, or combinations thereof) may comprise, or consists essentially of, linear alpha olefins. In an embodiment, the olefins of the olefin feedstock (whether aliphatic, linear or branched, acyclic, hydrocarbon, or combinations thereof) may comprise, or consists essentially of, hydrocarbon alpha olefins. In an embodiment, the olefins of the olefin feedstock may comprise, or consist essentially of, linear hydrocarbon alpha olefins; or alternatively, normal alpha olefins. In an embodiment, the olefins of the olefin feedstock (whether aliphatic, linear or branched, acyclic, hydrocarbon, alpha olefin, or any combination thereof) may comprise, or consist essentially of, mono-olefins. In some embodiments, the olefins of the olefin feedstock (whether aliphatic, linear or branched, acyclic, alpha olefin, or any combination thereof) may comprise, or consist essentially of, hydrocarbon mono-olefins. In an embodiment, the alpha olefins, the linear alpha olefin, the hydrocarbon alpha olefin may be mono-olefins.

In an embodiment, the olefin feedstock may comprise, or consist essentially of, olefins having at least 6 carbon atoms; alternatively, 8 carbon atoms; alternatively, at least 10 carbon atoms; or alternatively, at least 14 carbon atoms. In some embodiments, the olefin feedstock may comprise, or consist essentially of, olefins having from 8 to 50 carbon atoms; alternatively, from 8 to 30 carbon atoms; alternatively, from 8 to 20 carbon atoms; alternatively, from 10 to 50 carbon atoms; alternatively, from 10 to 30 carbon atoms; alternatively, from 10 to 20 carbon atoms; alternatively, from 14 to 30 carbon atoms; or alternatively, from 14 to 24 carbon atoms.

In an embodiment, the olefin feedstock can comprise a particular weight percentage of alpha olefins, hydrocarbon alpha olefins, linear alpha olefins, linear hydrocarbon alpha olefins, or normal alpha olefins. In some embodiment, the olefin feedstock can comprise greater than 60 weight percent alpha olefins, hydrocarbon alpha olefins, linear alpha olefins, linear hydrocarbon alpha olefins, or normal alpha olefins. In other embodiments, the olefin feedstock can comprise greater than 70, 80, 90, or 95 weight percent alpha olefins, hydrocarbon alpha olefins, linear alpha olefins, linear hydrocarbon alpha olefins, or normal alpha olefins. In other embodiments, the olefin feedstock can comprise from 60 to 99, 70 to 99, 80 to 98, or 90 to 98 weight percent alpha olefins, hydrocarbon alpha olefins, linear alpha olefins, linear hydrocarbon alpha olefins, or normal alpha olefins. In an embodiment, the alpha olefin, hydrocarbon alpha olefin, and linear alpha olefin of the olefin feedstock may be mono-olefinic. In a further embodiment, the olefin feedstock may consist essentially of a normal alpha olefin. The weight percentages of the alpha olefin, hydrocarbon alpha olefin, and linear alpha olefin also apply to any other type of alpha olefin, hydrocarbon alpha olefin, or linear alpha olefin (e.g. mono-olefinic, aliphatic, and acyclic, among others) described herein.

In an embodiment, the olefin feedstock can comprise, or consist essentially of, any olefin type described herein, any carbon number described herein, and/or any alpha olefin content (type and/or weight percentage) described herein. In some exemplary non-limiting combinations, the olefin feedstock can comprise linear alpha olefins having from 10 to 50 carbon atoms; alternatively, comprise greater than 90 weight percent hydrocarbon alpha olefins having from 10 to 30 carbon atoms; alternatively, comprise greater than 80 weight percent mono-olefinic hydrocarbon alpha olefins having from 10 to 30 carbon atoms; alternatively, comprise greater than 90 weight percent normal alpha olefins having from 10 to 20 carbon atoms; or alternatively, consist essentially of normal alpha olefins.

In an embodiment, the olefin feedstock can comprise, or consist essentially of, a normal alpha olefin. Suitable normal alpha olefins include those produced by ethylene oligomerization and/or by cracking heavy waxes (e.g. Fischer-Tropsch waxes). In some embodiments, the olefin feedstock can comprise, or consist essentially of, normal alpha olefins. One source of normal alpha olefins is Chevron Phillips Chemical Company LP, The Woodlands, Tex. Potential commercially available normal alpha olefin include, but are not necessarily limited to 1-hexene, 1-octene, 1-decene, 1-docecene, 1-tetradecene, 1-hexadecene, 1-octadecene, Alpha Olefin C₂₀₋₂₄, Alpha Olefin C₂₄₋₂₈, Alpha Olefin C₂₆₋₂₈, Alpha Olefin C₃₀₊ and/or Alpha Olefin C_{30+HA}. The normal alpha olefin may also be a Fischer-Tropsch product comprising a mixture of paraffin(s) and olefin(s) wherein the olefins meet the olefin feedstock parameters described herein. One source of Fischer-Tropsch waxes is Sasol, Johannesburg, South Africa.

The olefin feedstock may form part of an olefin feedstock composition comprising the olefin feedstock. For example, the olefin feedstock may be combined with a solvent or diluent to form an olefin feedstock composition. Such combinations may be utilized to improve the processing of the olefin feedstock in the isomerization process. In an embodiment, the olefin feedstock composition can comprise the olefin feedstock and a solvent or diluent. In some embodiments, the olefin feedstock composition can consist essentially of any olefin feedstock described herein; or alternatively, consists essentially of any olefin feedstock described herein and any solvent or diluent described herein. Solvents or diluents, which may be utilized in the olefin feedstock composition comprising the olefinic feedstock, are described herein. In other embodiments, the olefin feedstock composition comprising the olefin feedstock can be substantially devoid of solvent or diluent.

Generally the molecular sieve catalyst can be any molecular sieve catalyst that is capable of producing an olefin reactor effluent having the desired features (e.g. non-isomerized olefin content, isomerized olefin content, and skeletal isomerized olefin content, among others). However, depending upon isomerization reaction conditions (e.g. reaction temperatures, reaction time, and reaction pressure, among others), particular class(es) of molecular sieves may be favored in particular instances.

A variety of molecular sieve catalysts may be utilized in the isomerization process described herein may be described as having one or more particular features. Some features which may be utilized to describe the molecular sieve catalyst(s), either singly or in any combination, include the type of molecular sieve (zeolitic, non-zeolitic and/or specific type such as SAPO, SSZ, ZSM, among others), pore size, pore geometry, (e.g. major and minor axis width), and/or the presence or absence of particular metal(s) (e.g. group VIII metal). These features of the molecular sieve catalyst are independently described herein and may be utilized in any combination to describe the molecular sieve catalyst utilized to produce a particular isomerized olefin described herein.

In an aspect, the molecular sieve catalyst may comprise pores having a pore size ranging from 2 to 10 angstroms. In other embodiments, the molecular sieve catalyst may comprise pores having a pore size ranging from 4 to 8 angstrom; alternatively, ranging from 5 to 7 angstroms; or alternatively, ranging from 5.3 to 6.5 angstroms.

The pore size of the molecular sieves can be measured using standard adsorption techniques and hydrocarbonaceous compounds of known minimum kinetic diameters. See Breck, Zeolite Molecular Sieves, 1974 (Chapter 8): Anderson et al., J. Catalysis 58, 114 (1979); and U.S. Pat. No. 4,440,871, the pertinent disclosures of which are incorporated herein by reference.

In performing adsorption measurements to determine pore size, standard techniques are used. Generally, it is convenient to consider a particular molecule as excluded if it does not reach at least 95% of its equilibrium adsorption value on the molecular sieve in less than about 10 minutes (p/po=0.5:25.degree.).

In an aspect, the pore of the molecular sieve catalyst may have a particular geometry. Generally, the molecular sieve catalyst may comprise generally oval, one-dimensional pores having a minor axis and a major axis. In an embodiment, the molecular sieve catalyst comprises oval one-dimensional pores having a minor axis ranging from 2 to 6 angstroms and a major axis ranging group 3 to 9 angstroms; alternatively, having a minor axis ranging from 3 to 5 angstroms and a major axis ranging group 4 to 8 angstroms; or alternatively, having a minor axis ranging from 4 to 5 angstroms and a major axis ranging from 5 to 7.5 angstroms; or alternatively, a minor axis ranging from 4.2 angstroms to 4.8 angstroms and a major axis ranging from 5.4 angstroms to 7.0 angstroms.

In an embodiment, the molecular sieve catalyst may be a SAPO molecular sieve, a SSZ molecular sieve, or ZSM molecular sieve. In some embodiment, the molecular sieve catalyst may be a SAPO molecular sieve; alternatively, a SZM molecular sieve; or alternatively, a ZSM molecular sieve. In an embodiment, the molecular sieve catalyst may be SAPO-11, SAPO-31, SAPO-41, SSZ-32, ZSM-22, ZSM-23, ZSM-35, or combination thereof. In some embodiments, the molecular sieve catalyst may be SAPO-11, SAPO-31, SAPO-41, or combinations thereof. In other embodiments, the molecular sieve catalyst may be SAPO-11; alternatively SAPO-31; or alternatively, SAPO-41. In an embodiment, the molecular sieve catalyst may be SSZ-32. In an embodiment, the molecular sieve catalyst may be ZSM-22, ZSM-23, ZSM-35, or combinations thereof. In some embodiments, the molecular sieve catalyst may be ZSM-22; alternatively, ZSM-23; or alternatively, ZSM-35. The SAPO-11, SAPO-31, SAPO-41, SSZ-32, ZSM-22, ZSM-23, ZSM-35 molecular sieves are disclosed in U.S. Pat. No. 5,246,566 to Miller, U.S. Pat. No. 5,252,527 to Zones, U.S. Pat. No. 4,076,842 to Plank et al., U.S. Pat. No. 4,440,871 to Lok et al., U.S. Pat. No. 4,556,477, and U.S. Pat. No. 4,016,245, and U.S. Pat. No. 4,107,195. The full disclosure of these patents is incorporated herein by reference.

In an aspect, a useful molecular sieve is commonly known as a “non-zeolitic molecular sieve.” Non-zeolitic molecular sieves are three-dimensional microporous crystalline structures containing AlO₂ and PO₂ oxide units. The non-zeolitic molecular sieves may further contain silicon and/or one or more metals other than aluminum which form tetrahedral coordinate oxide linkages with aluminum and/or phosphorous in a crystalline framework. In some embodiments the non-zeolitic molecular sieves may comprise MO₂, AlO₂, and PO₂ tetrahedrally bound structural oxide units, where M represents at least one element, which forms oxides in tetrahedral coordination with Al₂, and PO₂ units. In other embodiments, the non-zeolitic molecular sieves comprise MO₂, SiO₂, AlO₂, and PO₂ oxide units, where M represents an element that form oxides in tetrahedral coordination with AlO₂ and PO₂ units. In an embodiment, the metal, M, of the non-zeolitic molecular sieve comprising MO₂, AlO₂, and PO₂ or MO₂, SiO₂, AlO₂, and PO₂ tetrahedrally bound structural oxide units, may be arsenic, beryllium, boron, chromium, cobalt, gallium, germanium, iron, lithium, magnesium, manganese, silicon, titanium, vanadium, and zinc.

In an embodiment, the non-zeolitic molecular sieve may be an aluminophosphate molecular sieve. In other embodiments the non-zeolitic molecular sieve may be a silicoaluminophosphate molecular sieve.

Non-zeolitic molecular sieve are described in the literature. Aluminophosphate non-zeolitic molecular sieves are described in U.S. Pat. No. 4,310,440. Silicoaluminophosphate non-zeolitic molecular sieves comprising tetrahedrally coordinated AlO₂, PO₂, and SiO₂ structural units are described in U.S. Pat. Nos. 4,440,871, 4,943,424, and 5,087,347. U.S. Pat. No. 4,567,029 describes non-zeolitic molecular sieves where M is selected from the group consisting of magnesium, manganese, zinc, and cobalt. U.S. Pat. No. 4,913,799 describes non-zeolitic molecular sieves where M is selected from the group consisting of arsenic, beryllium, boron, chromium, cobalt, gallium, germanium, iron, lithium, magnesium, manganese, silicon, titanium, vanadium, and zinc. U.S. Pat. No. 4,973,785 describes non-zeolitic molecular sieves comprising tetrahedrally bound structural units comprising MO₂, SiO₂, AlO₂, and PO₂ oxide units, where M represents an element which forms oxides in tetrahedral coordination with AlO₂ and PO₂ units. The disclosures of each of these cited patents are incorporated herein by reference in their entirety.

Unless otherwise specified, the molecular sieve catalyst may be a zeolitic molecular sieve or a non-zeolitic molecular sieve. Persons of ordinary skill in the art recognize molecular sieves that are zeolitic or non-zeolitic.

In an embodiment, the molecular sieve catalyst(s) (zeolitic or non-zeolitic) may comprise a transition metal. In further embodiments, the molecular sieve catalyst(s) may comprise a group VIII metal. In yet other embodiments, the molecular sieve catalyst(s) may comprise platinum or palladium; alternatively, platinum; or alternatively, palladium.

In an embodiment, the molecular sieve catalyst(s) may be substantially free platinum; alternatively, palladium; or alternatively platinum and palladium. In some embodiments, the molecular sieve catalyst(s) may be substantially free of a group VIII metal. In other embodiments, the molecular sieve catalyst(s) may be substantially free of a transition metal.

Generally, the method(s) for producing isomerized olefins may be conducted using reaction conditions which can provide an olefin product having the desired features. Isomerization reaction conditions which may be utilized to form a desired olefin product may include the reaction temperature, the weight hourly space velocity, the reaction pressure, the conversion of the olefin feedstock to an isomerized olefin, the amount of skeletally isomerized olefin found in the reactor effluent, and the presence or absence of a solvent or diluent, among others. The isomerization reaction conditions are independently described herein and may be used in the combination(s) necessary to produce a reactor effluent having the desired features. Furthermore, the reaction temperature, weight hourly space velocity, and reaction pressure may also be referred to as the isomerization reaction temperature, isomerization weight hourly space velocity, and isomerization reaction pressure, respectively.

In an embodiment, the isomerization reaction temperature may range from 80 to 220° C. In some embodiments, the isomerization reaction temperature may range form 100 to 200° C.; alternatively, ranging from 110 to 190° C.; or alternatively, 120 to 180° C.

In an embodiment the weight hourly space velocity may range from 0.01 to 1.0. In some embodiments, the weight hourly space velocity may range from 0.02 to 0.7; alternatively 0.02 to 0.5; alternatively, ranging from 0.03 to 0.3; alternatively, ranging from 0.04 to 0.2; alternatively, ranging from 0.04 to 0.18; or alternatively, ranging from 0.05 to 0.15.

One of ordinary skill in the art recognizes that there is a relationship between the isomerization reaction temperature and the weight hourly space velocity. Generally, to obtain an isomerized product having equivalent features, an increase in the weight hourly space velocity will require an increase in the isomerization reaction temperature. Additionally, one of ordinary skill in the art recognizes that as the time the molecular sieve ages, the isomerization reaction temperature must be increased and/or the weight hourly space velocity must be decreased to maintain an olefin reactor effluent having the desired features.

Generally, the isomerization reaction is performed at a temperature which maintains the isomerization reaction solution in a processable state. Depending on the olefin feedstock and/or the isomerized product, the olefin isomerization conditions may not be able to maintain the isomerization reaction solution in a processable state. In these cases, the isomerization reaction solution may utilize a solvent or diluent maintain the reaction solution in a processable state. Applicable solvents or diluents are described herein and may form part of the isomerization reaction solution. It will also be appreciated that a solvent or diluent may be utilized even if the isomerization reaction conditions alone can maintain the isomerization reaction solution in a processable state.

The term “processable state” refers to a solution which can be stirred, pumped, and/or is sufficiently fluid to flow through a column. Consequently, an isomerization reaction solution in a “processable state” does not necessarily refer to isomerization reaction solution wherein all materials are in the liquid and/or gaseous state. For example, the processable solution may comprise solid (non-liquid or undissolved) particles (e.g. wax) which do not prevent the ability to stir and/or pump the isomerization reaction solution or impede the isomerization reaction solution flow through a column.

Generally, the reaction pressure of the olefin isomerization process may be any reactor pressure compatible with the olefin feedstock, process(es), and equipment. In an embodiment, the reaction pressure may be maintained at atmospheric pressure. In some embodiments, the reaction pressure may be maintained at a pressure greater than atmospheric pressure. In other embodiments, the reaction pressure may be maintained within 20 psig of atmospheric pressure. In further embodiments, the reaction pressure may range from atmospheric pressure to 1000 psig; alternatively, from atmospheric pressure to 500 psig; or alternatively from atmospheric pressure to 100 psig. In particular embodiments, the reaction pressure may be maintained at a pressure greater than the pressure that maintains olefin feedstock (or reaction solution) in a liquid state at the reaction temperature employed. In some other embodiments, the reaction pressure for the isomerization process may be maintained at a pressure ranging from a pressure that maintains olefin feedstock in a liquid state at the reaction temperature employed and 1000, 500, or 100 psig.

Generally, the isomerization reaction solution comprises the materials which are contacted in any reactor described herein. In an embodiment, the isomerization reaction solution comprises the olefin feedstock and the molecular sieve catalyst. In some embodiments, the reaction solution comprises the olefin feedstock, the molecular sieve catalyst, and a solvent or diluent. In other embodiments, the isomerization reaction solution consists essentially of the olefin feedstock and the molecular sieve catalyst. In yet other embodiments, the isomerization reaction solution consists essentially of the olefin feedstock, the molecular sieve catalyst, and a solvent or diluent. In further embodiments, the isomerization reaction solution is substantially devoid of solvent or diluent. Persons with ordinary skill in the art will recognize which solvent(s) or diluent(s) classes and/or specific solvent(s) or diluent(s) are compatible with a particular molecular sieve catalyst class or specific molecular sieve catalyst.

Generally, the isomerization reaction can occur in any reactor capable of allowing the isomerization reaction to take place. In an embodiment, the isomerization may take place in a fixed bed reactor; alternatively, in a continuous stirred tank reactor. In an embodiment, the isomerization may be performed in a continuous process; or alternatively, in a batch process. In an embodiment, the isomerization may be carried out as a continuous process employing a fixed bed reactor; or alternatively, one or more continuous stirred tank reactors.

The solvent or diluent which may be utilized for the olefin feedstock composition comprising the olefin feedstock, and/or the reaction solution can comprise, or consist essentially of, a hydrocarbon, a halogenated hydrocarbon, or combinations thereof. In some embodiments, the solvent or diluent, can comprise, or consist essentially of, a hydrocarbon; or alternatively, a halogenated hydrocarbon. In some embodiments, the hydrocarbon solvent or diluent can be a saturated hydrocarbon; or alternatively, an aromatic hydrocarbon.

In an embodiment, the solvent or diluent can comprise, or consist essentially of, a C₄ to C₂₀ saturated hydrocarbon; or alternatively, a C₅ to C₁₀ saturated hydrocarbon. In some embodiments, the solvent or diluent can comprise, or consist essentially of, a C₆ to C₂₀ aromatic hydrocarbon; or alternatively, C₆ to C₁₀ aromatic hydrocarbon. In some embodiments, the solvent or diluent can comprise, or consist essentially of, a C₁ to C₁₅ halogenated hydrocarbon; alternatively, C₁ to C₁₀ halogenated hydrocarbon; or alternatively, C₁ to C₅ halogenated hydrocarbon.

Suitable saturated hydrocarbon solvent(s) or diluent(s) can include butane, isobutane, pentane, n-hexane, hexanes, cyclohexane, n-heptane, n-octane, or mixtures thereof; or alternatively, n-hexane, hexanes, cyclohexane, n-heptane, n-octane, or mixtures thereof. Suitable aromatic hydrocarbon solvent(s) or diluent(s) can include benzene, toluene, mixed xylenes, ortho-xylene, meta-xylene, para-xylene, ethylbenzene, or mixtures thereof. Suitable halogenated solvent(s) or diluent(s) can include carbon tetrachloride, chloroform, methylene chloride, dichloroethane, trichloroethane, chlorobenzene, or dichlorobenzene, or mixtures thereof.

Generally, the reactor effluent comprises an isomerized product. The isomerized product may further comprise isomerized olefins and/or skeletally isomerized olefins. The reactor effluent may further comprise other elements which were charged to the isomerization reactor (e.g. molecular sieve catalyst, and solvent or diluent, among others). Generally, the olefin reactor effluent consists of all olefins which exit the reactor. The olefins which exit the reactor may include non-isomerized olefin, isomerized olefin, and/or skeletally isomerized olefin. The non-isomerized olefin, isomerized olefin, and skeletally isomerized olefins, which may be present in the olefin reactor effluent, are independently described herein. Additionally, the quantities of non-isomerized olefin, isomerized product, isomerized olefin, skeletally isomerized olefin, which may be found in the olefin reactor effluent, are independently described herein and may be utilized in any combination to describe the olefin reactor effluent of the method(s) described herein.

In an embodiment, the olefin reactor effluent may comprise an isomerized product. In some embodiments, the olefin reactor effluent may comprise an isomerized product and non-isomerized olefin. Generally the isomerized product may comprise, or consist essentially of, isomerized olefins and skeletally isomerized olefins. Alternatively, the isomerized product may comprise, or consist essentially of, linear and/or branched olefins.

Within the definitions of the present disclosure, one should recognize that an isomerized olefin is not necessarily equivalent to a linear olefin and that skeletally isomerized olefin is not necessarily equivalent to branched olefin. Some useful olefin feedstocks for the herein described isomerization methods, such as commercially available normal alpha olefins, may contain branched olefins wherein the branches may occur on the carbon atom of the olefin double bond (e.g. a vinylidene) or a branch on a carbon atom which is not part of the olefin double bond. In some instances, the isomerization methods described herein may change position of the olefin bond in the branched olefin to create an isomerized product that is branched without a rearrangement of carbon atoms. For example, the olefin double bond of 2-ethyl-1-decene (a vinylidene) may be isomerized to 3-methyl-2-tridecene or 3-methyl-3-tridecene without a rearrangement of carbon atoms. In such an instance, the 3-methyl-2-tridececene and 3-methyl-3-tridecene represent branched isomerized olefin but does not represent a skeletally isomerized olefin because no rearrangement of carbon atoms has occurred.

Generally, the branched products of the isomerization methods described herein are indistinguishable from each other. However, when determining the weight percentage of skeletally isomerized olefins, only the weight percent of products having branches in excess of the weight percent of olefins having branches in the olefin feedstock are considered to be skeletally isomerized. Consequently, when the feedstock of the isomerization process contains branched materials, the amount of skeletally isomerized product is the difference between the amount of branched product in the olefin reactor effluent and the amount of branched product in the feedstock. For example, the amount of skeletally isomerized olefin is the difference between the amount of branched olefins in the olefin reactor effluent and the amount of branched olefin in the olefin feedstock.

The amount of branched olefins present in the olefin feedstock and olefin reactor effluent can be determined using various methods. One of the easiest methods for determining the amount of branched olefin in an olefin feedstock and olefin reactor effluent is to hydrogenate the olefin feedstock and the olefin reactor effluent to saturated compounds and then analyze the hydrogenated products by gas chromatography (hereafter GC) using a GC column and method capable of separating linear and branched saturated molecules having the same carbon number. GC columns which may be utilized for the GC analysis of the hydrogenated product include the HP Ultra-1 line of capillary columns and the HP-5 line of capillary columns. Persons of ordinary skill in the art know other GC columns which are capable of separating linear and branched saturated products having the same carbon numbers. Persons of ordinary skill in the art would also know how to adjust GC analysis conditions for the particular carbon numbers present in the olefin feedstock or olefin reactor effluent.

In an embodiment, the olefin reactor effluent may comprise greater than 85 weight percent isomerized product. In some embodiments, the olefin reactor effluent may comprise greater than 90, 92, 94, 95, or 96 weight percent isomerized product.

In an embodiment, the olefin reactor effluent comprises less than 10 weight percent non-isomerized olefin. Alternatively, the olefin reactor effluent comprises less than 8, 6, 5, 4 weight percent non-isomerized olefin. Generally, the non-isomerized olefin may be any olefin of the olefin feedstock described herein (e.g. alpha olefin, hydrocarbon alpha olefin, linear alpha olefin, normal alpha olefin, among others).

In an embodiment, the isomerized product of the olefin reactor effluent may have less than 10, 8, 7, or 6 weight percent skeletally isomerized olefins. In some embodiments, the isomerized product of the olefin reactor effluent may have less than 5 weight percent skeletally isomerized olefins. In another embodiment, the isomerized product of the olefin reactor effluent may have less than 4.75, 4.5, 4.0, or 3.75 weight percent skeletally isomerized olefin.

In an embodiment, the isomerized product of the olefin reactor effluent may have greater than 70 weight percent linear internal olefins. In other embodiments, the isomerized product of the olefin reactor effluent may have greater than 75, 80, 85, or 90 weight percent linear internal olefins. In another embodiment, the isomerized product of the olefin reactor effluent may have from 75 to 98 weight percent linear internal olefins. In further embodiments, the isomerized olefin product of the olefin reactor effluent may have from 80 to 97, from 85 to 96, or from 90 to 96, linear internal olefins.

In an embodiment, the isomerized product of the olefin reactor effluent may have less than 30 weight percent branched olefins. In some embodiments, the isomerized product of the olefin reactor effluent may have less than 25, 20, 15, or 10 weight percent branched olefins. In other embodiments, the isomerized product of the olefin reactor effluent may have from 2 to 25 weight percent branched olefins. In a further embodiment, the isomerized product of the olefin reactor effluent may have from 3 to 20, from 4 to 15, or from 4 to 10 weight percent branched olefins.

In an aspect, the methods described herein may be utilized to control the skeletally isomerized olefin content of the olefin reactor effluent. Generally, the method for controlling the skeletally isomerized olefin content of the olefin reactor effluent comprises selecting the isomerization reaction conditions (e.g. temperature and weight hourly space velocity) to obtain an olefin reactor effluent having the desired skeletally isomerized olefin content. In an embodiment, the method for isomerizing olefins comprises: 1) controlling a skeletally isomerized olefin content of an olefin reactor effluent by selecting isomerization reaction parameters including: a) a molecular sieve catalyst, and b) isomerization reaction conditions; 2) contacting an olefin feedstock and the molecular sieve catalyst; and 3) isomerizing the olefin feedstock in a reactor at the isomerization conditions to produce an olefin reactor effluent having a desired skeletally isomerized olefin content. In another embodiment, the method for isomerizing olefins comprises: 1) contacting an olefin feedstock and a molecular sieve, and 2) reacting the olefin feedstock in a reactor at specific isomerization reaction conditions to produce an olefin reactor effluent having a desired skeletally isomerized olefin content.

Molecular sieve catalysts, which may be utilized for controlling the skeletally isomerized olefin content of an olefin reactor effluent in a process to isomerize olefins, are independently described herein and may be utilized in any combination to describe the isomerization conditions to control the skeletally isomerized olefin content of the olefin reactor effluent. Isomerization reaction conditions, which may be utilized to produce a desired olefin reactor effluent and/or skeletally isomerized olefin content, are independently described herein and may be utilized in any combination to describe the isomerization conditions to control the skeletally isomerized olefin content of the olefin reactor effluent. Features describing the olefin reactor effluent, non-isomerized olefin, isomerized product, isomerized olefin, and skeletally isomerized olefin are independently described herein and may be utilized in any combination to describe the product of the method(s) to control the skeletally isomerized olefin content of the olefin reactor effluent. Generally, the olefin feedstock utilized in the method(s) of controlling the skeletally isomerized olefin content of an olefin reactor effluent in a process to isomerize olefins may be any olefin feedstock described herein.

In an embodiment, an olefin reactor effluent having less than 10 weight percent non-isomerized olefins and less than 5 weight percent skeletally isomerized olefins may be produced by contacting an olefin feedstock with a molecular sieve catalyst at a reaction temperature ranging from 100 to 200° C. and a weight hourly space velocity ranging from 0.2 to 0.7. In another embodiment, an olefin reactor effluent having less than 10 weight percent non-isomerized olefin and 10 weight percent skeletally isomerized olefin may be produced by contacting an olefin feedstock with a molecular sieve catalyst at a reaction temperature ranging from 100 to 200° C. and a weight hourly space velocity ranging from 0.04 to 018.

In an embodiment, the molecular sieve catalyst utilized for controlling the amount of skeletally isomerized olefin in the olefin reactor effluent of a olefin isomerization process may have any pore size and/or pore geometry describe herein. In some embodiments, the molecular sieve catalyst utilized for controlling the amount of skeletally isomerized olefin in the olefin reactor effluent of an olefin isomerization process may a SAPO, SSZ, or ZSM molecular sieve. In other embodiments, the molecular sieve catalyst utilized for controlling the amount of skeletally isomerized olefin in the olefin reactor effluent of an olefin isomerization process may be a SAPO molecular sieve; alternatively, a SSZ molecular sieve; or alternatively, a ZSM molecular sieve.

While particular embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Use of the term “optionally” with respect to any element is intended to mean that the subject element is required, or alternatively, is not required. Both alternatives are intended to be within the scope. Use of broader terms such as “comprises,” “includes,” “has” and “having,” etc. should be understood to provide support for narrower terms such as “consisting essentially of,” “consisting of,” “comprised substantially of,” etc.

The scope of protection is not limited by the description set out within but is only limited by the claims which follow, that scope including all equivalents of the subject matter of the claims. Each and every claim is incorporated into the specification as an embodiment of the present invention. Consequently, the claims are a further description and are an addition to the particular embodiments of the present invention. The discussion of a reference within this application is not an admission that it is prior art to the present invention(s), especially any reference that may have a publication date after the priority date of this application. The disclosures of all patents, patent applications, and publications cited herein are hereby incorporated by reference, to the extent that they provide exemplary, procedural or other details supplementary to those set forth herein.

The following examples are included to demonstrate specific embodiments of the invention(s). Those of skill in the art should appreciate that the techniques disclosed in the examples represent techniques discovered to function well in the practice of the invention. However, in light of the present disclosure, those of skill in the art will appreciate the changes that can be made in the specific disclosed embodiments and still obtain similar results that do not depart from the spirit and scope of the invention.

### EXAMPLES

In the examples, commercially available normal alpha olefins obtained from Chevron Phillips Chemical Company, LP, were subjected to isomerization using a molecular sieve catalyst to produce an isomerized product.

### Isomerization Reaction

A mixture containing 35 weight percent 1-tetradecene, 35 weight percent 1-hexadecene, and 30 percent 1-octadecene was passed over a SAPO-11 catalyst at 150° C. at a weight hourly space velocity of 0.1. The olefin reactor effluent contained 3.86 alpha olefin as determined by FTIR. Hydrogenation and GC analysis of the normal alpha olefin feedstock indicated the presence of 9.7 weight percent branched olefin in the normal alpha olefin feedstock. Hydrogenation and GC analysis of the olefin reactor effluent indicated the presence of 11.03 weight percent branched product. By the difference in these numbers, the isomerized product contained 1.33 weight percent skeletally isomerized olefins.

The hydrogenation and analysis of the normal alpha olefin feedstock and olefin reactor effluent are described herein.

### Hydrogenation of the Normal Alpha Olefin and Olefin Reactor Effluent

A sample of the 1-tetradecene/1-hexadecene/1-octadecene olefin feedstock or isomerization reactor effluent (approximately 1 mL) was dissolved in n-tridecane (approximately 10 g). This mixture was then added to of a 10 weight percent palladium on carbon hydrogenation catalyst (approximately 0.1 gram) and contacted with a stream of hydrogen gas at atmospheric pressure and 40° C. for 3 hours. The hydrogenated olefin feedstock or isomerization reactor effluent was then separated from hydrogenation catalyst by filtration. A sample of the filtrate was than analyzed using the following GC analysis procedure. Persons having ordinary skill in the art would recognize that the tridecane solvent may be substituted with another appropriate solvent if it would interfere with the GC analysis of the hydrogenated olefin feedstock or isomerization reactor effluent for other olefin feedstocks and isomerization reactor effluents.

### Hydrogenated Normal Alpha Olefin and Olefin Reactor Effluent GC Analysis

The filtrate from the hydrogenation of the normal alpha olefin feedstock and isomerization reactor effluent was analyzed using Gas Chromatography (GC). The GC analyses were conducted on Hewlett Packard HP6890 System, using a 12 m×0.20 mm×0.33 μm HP-5 column. The analysis was performed using a split injection with a 10 ml/min helium carrier gas flow rate. The injection port temperature was 275° C. The detector for the analysis was a flame ionization detector operated at 325° C. with a H₂ flow of 40 mL/min, an air flow of 450 mL/min, and a helium makeup flow of 45 mL/min. The GC analysis oven temperature was programmed for an initial temperature of 100° C. for 2 minutes, a first temperature ramp of 8° C./min to 185° C. at a rate of immediately followed by a second temperature ramp of 20° C. to 320° C. and a hold time of 6 minutes at 320° C. The sample size injected onto the GC analysis column was 0.5 microliter. The quantities of the linear and branched materials in the hydrogenated olefin feedstock and isomerization reactor effluent were determined by integrating the linear and branched peaks in the GC chromatogram using techniques know to those with ordinary skill in the art.

All of the compositions and methods disclosed and claimed herein can be made and executed without undue experimentation in light of the present disclosure. While the compositions and methods of this invention have been described in terms of particular embodiments, it will be apparent to those of skill in the art that variations may be applied to the compositions and methods and in the steps or in the sequence of steps of the methods described herein without departing from the concept, spirit and scope of the invention as defined by the appended claims.

What is claimed is:

1. A method for producing isomerized olefins comprising:
a) contacting
i) an olefin feedstock comprising linear alpha olefins having at least 8 carbon atoms; and
ii) a molecular sieve catalyst substantially free of platinum and palladium; and
b) isomerizing the linear alpha olefins in a reactor at reaction conditions comprising a weight hourly space velocity ranging from 0.02 to 0.7 to form an olefin reactor effluent comprising less than 10 weight percent linear alpha olefins and an isomerized product having less than 5 weight percent skeletally isomerized olefins.

2. The method of claim 1, wherein the olefin reactor effluent comprises less than 5 weight percent linear alpha olefin.

3. The method of claim 1, wherein the olefin reactor effluent comprises less than 4 weight percent linear alpha olefin and the isomerized product has less than 4.5 weight percent skeletally isomerized olefin.

4. The method of claim 1, wherein the molecular sieve catalyst comprises pores having a pore size ranging from 4 to 8 angstroms.

5. The method of claim 1, wherein the molecular sieve catalyst comprises oval one-dimensional pores having a minor axis ranging from 2 to 6 angstroms and a major axis ranging group 3 to 9 angstroms.

6. The method of claim 1, wherein the molecular sieve catalyst is selected from the group consisting of SSZ-32, ZSM-23, ZSM-22, ZSM-35, SAPO-11, SAPO-31, SAPO-41, or combinations thereof.

7. The method of claim 1, wherein the reaction conditions further comprise a reaction temperature ranging from 100 to 200° C.

8. The method of claim 1, wherein the weight hourly space velocity ranges from 0.03 to 0.3 and the reaction conditions further comprise a reaction temperature ranging from 100 to 200° C.

9. The method of claim 1, wherein the linear alpha olefins have from 14 to 30 carbon atoms.

10. The method of claim 9, wherein the olefin feedstock comprises greater than 90 weight percent mono-olefinic linear alpha olefins.

11. The method of claim 1, wherein the olefin feedstock consists essentially of normal alpha olefins.

12. The method of claim 1, wherein the olefin feedstock comprises greater than 90 mole percent normal alpha olefins having from 14 to 30 carbon atoms; the molecular sieve catalyst is selected from the group consisting of SAPO-11, SAPO-31, SAPO-41, and combinations thereof; the molecular sieve catalyst is substantially free of a transition metal; the weight hourly space velocity ranges from 0.04 to 0.2; the reaction conditions further comprise a reaction temperature ranging from ranging from 120 to 180° C.; and the olefin reactor effluent comprises less than 4 percent normal alpha olefin and the isomerized product has less than 4 weight percent skeletally isomerized olefins.

13. A method for producing an isomerized olefin comprising:
a) contacting
i) an olefin feedstock comprising linear alpha olefins having at least 8 carbon atoms; and
ii) a molecular sieve catalyst substantially free of a platinum and palladium; and
b) isomerizing the linear alpha olefins in a reactor at reaction conditions comprising a weight hourly space velocity ranging from 0.04 to 0.18 to form an olefin reactor effluent comprising less than 10 weight percent linear alpha olefins and an isomerized product having less than 10 weight percent skeletally isomerized olefin.

14. The method of claim 13, wherein the olefin reactor effluent comprises less than 5 weight percent linear alpha olefin.

15. The method of claim 13, wherein the olefin reactor effluent comprises less than 4 weight percent linear alpha olefin and the isomerized product has less than 7 weight percent skeletally isomerized olefin.

16. The method of claim 13, wherein the molecular sieve catalyst comprises pores having a pore size ranging from 4 to 8 angstroms.

17. The method of claim 13, wherein the molecular sieve catalyst comprises oval one-dimensional pores having a minor axis ranging from 2 to 6 angstroms and a major axis ranging group 3 to 9 angstroms.

18. The method of claim 13, wherein the molecular sieve catalyst is selected from the group consisting of SSZ-32, ZSM-23, ZSM-22, ZSM-35, SAPO-11, SAPO-31, SAPO-41, or combinations thereof.

19. The method of claim 13, wherein the reaction conditions further comprise a reaction temperature ranging from 100 to 200° C.

20. The method of claim 13, wherein the WHSV ranges from 0.05 to 0.15 and the reaction conditions further comprise a reaction temperature ranging from 100 to 200° C.

21. The method of claim 13, wherein the linear alpha olefins have from 14 to 30 carbon atoms.

22. The method of claim 21, wherein the olefin feedstock comprises greater than 90 weight percent mono-olefinic linear alpha olefins.

23. The method of claim 13, wherein the olefin feedstock consists essentially of normal alpha olefins.

24. The method of claim 13, wherein the olefin feedstock comprises greater than 90 mole percent normal alpha olefins having from 14 to 30 carbon atoms; the molecular sieve catalyst is selected from the groups consisting of SAPO-11, SAPO-31, SAPO-41, and combinations thereof; the molecular sieve catalyst is substantially free of a transition metal; the weight hourly space velocity ranges from 0.05 to 0.15 and the reaction conditions comprise a reaction temperature ranging from ranging from 120 to 180° C.; and the reactor effluent comprises less than 5 percent normal alpha olefin and the isomerized product has less than 6 weight percent skeletally isomerized olefins.
